(19) United States
(12) Patent Application Publication
(10) Pub. No.: US 2012/0095122 A1
(43) Pub. Date: Apr. 19, 2012

(54) POLYURETHANE MICROCELLULAR ELASTOMER, METHOD FOR PREPARING SAME AND USE THEREOF

(75) Inventors: Zhiping Zhou, Shanghai (CN); Zhong Cao, Shanghai (CN); Yide Liang, Shanghai (CN); Yuedong Zhang, Shanghai (CN); Jen Chieh Roy Lin, Singapore (SG)

(73) Assignee: Bayer MaterialScience AG, Leverkusen (DE)

(21) Appl. No.: 13/263,617

(22) PCT Filed: Mar. 31, 2010
(86) PCT No.: PCT/EP2010/002047

§ 371 Date: Jan. 9, 2012

(30) Foreign Application Priority Data
Apr. 10, 2009 (CN) 200910049090.2

(51) Int. Cl. C08G 18/66; C07C 265/04; C07C 265/08; C08J 9/00

(52) U.S. Cl. 521/159; 558/266; 558/275; 558/276

(57) ABSTRACT

The present invention relates to polyurethane, and specifically relates to a polyurethane microcellular elastomer, a method for preparing the same, and the use thereof. The polyurethane microcellular elastomer is the reaction product of (1) an isocyanate-terminated prepolymer obtained by reacting an isocyanate with a first polyol, (2) a second polyol, (3) a chain extender and (4) a catalyst, wherein the first polyol and/or second polyol comprise at least one polycarbonate polyol obtained by reacting carbon dioxide with an alkene oxide. This method of preparing polyurethane microcellular elastomer can not only reduce the demand for raw materials derived from petrochemicals, but also realize the utilization of the “greenhouse gas” carbon dioxide.

### TECHNICAL FIELD

The present invention relates to polyurethane, and specifically relates to a polyurethane microcellular elastomer, a method for preparing the same, and the use thereof.

### BACKGROUND ART

Polyurethane microcellular elastomer is a material widely used in industry due to its excellent physical performance, such as high abrasion resistance, high tear strength, good flex property, and low density. The elastomer is often used in the footwear and automotive industries. Especially in the footwear industry, shoe soles made from polyurethane microcellular elastomer possesses advantages such as light weight, slip resistance, high mechanical strength, abrasion resistance, and oil resistance.

Presently, there are two kinds of polyurethane microcellular elastomers for shoe soles, namely polyester-type polyurethane microcellular elastomer and polyether-type polyurethane microcellular elastomer. Polyester-type polyurethane microcellular elastomer has good mechanical properties, but is too weak to withstand hydrolysis or microbial attack. Furthermore, it exhibits unsatisfactory physical behaviors at low temperatures. Therefore, the processing procedure is complicated. Polyether-type polyurethane microcellular elastomer is hydrolysis stable with good cold flex properties. However, it has relatively poor mechanical properties.

In the prior art, polyurethane microcellular elastomer is prepared by one step or multi-step reactions between isocyanates, polyester polyols and/or polyether polyols. CN1982351A discloses a method for preparing NDI polyester-type polyurethane microcellular elastomer. CN101328254A discloses a method for preparing polyester-type polyurethane microcellular elastomer by adding polycarbonate polyols. CN1428359A discloses a method for preparing polyether-type polyurethane microcellular elastomer by a semi-prepolymer method. CN101165094A discloses a method for preparing a polyether-type polyurethane microcellular elastomer by using high reactivity, low unsaturation polyether polyols. In addition, CN1986592A discloses a method for preparing a polyether-type polyurethane microcellular elastomer possessing improved mechanical behaviors by adding polymer polyols and organic silicon surfactants. However, in all of the above-mentioned methods, as the raw materials, all of the polyester polyols, polyether polyols and polycarbonate polyols are obtained through petrochemical routes. It is well known that petrochemicals are non-renewable resources. Therefore, intensive efforts have been made in exploring alternative routes which will partially or completely avoid the use of raw materials from petrochemical processes.

### CONTENTS OF INVENTION

The objective of the present invention is to provide a polyurethane microcellular elastomer. In one embodiment of the present invention, the polyurethane microcellular elastomer comprises the reaction product of:
a) an isocyanate-terminated prepolymer, said isocyanate-terminated prepolymer being a reaction product of an isocyanate and a first polyol;
b) a second polyol; and
c) a chain extender having an average molecular weights of less than or equal to 800;
   wherein, said first polyol and/or second polyol comprise(s) at least one polycarbonate polyol obtained by reacting carbon dioxide with an alkene oxide, wherein said at least one polycarbonate polyol has the general formula (I):

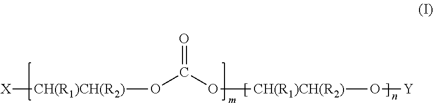

wherein,
R1 and R2 are independently selected from the group consisting of hydrogen atom, methyl, ethyl, and phenyl;
m is a natural number;
n is 0 or a natural number; and
X and Y are independently a terminal group comprising hydroxyl.

Another objective of the present invention is to provide an isocyanate-terminated prepolymer for preparing the polyurethane microcellular elastomer of the present invention. In another embodiment of the present invention, the isocyanate-terminated prepolymer is the reaction product of an isocyanate with a first polyol, wherein said first polyol comprises at least one polycarbonate polyol obtained by reacting carbon dioxide with an alkene oxide, wherein said at least one polycarbonate polyol has the general formula (I)

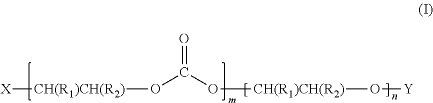

wherein,

R1 and R2 are independently selected from the group consisting of hydrogen atom, methyl, ethyl, and phenyl;

m is a natural number;

n is 0 or a natural number; and

X and Y are independently a terminal group comprising hydroxyl.

In the above mentioned reaction components, the amount of the polycarbonate polyol, obtained by reacting carbon dioxide and alkene oxide, in the first polyol is in the range of from 5 to 100 weight %, based on 100 weight % of the first polyol.

In the above mentioned reaction components, the NCO content of the isocyanate-terminated prepolymer is in the range of from 5 to 25 weight %, based on 100 weight % of the isocyanate-terminated prepolymer.

In the above mentioned reaction components, the amount of the polycarbonate polyol, obtained by reacting carbon dioxide and alkene oxide, in the second polyol is in the range of from 5 to 100 weight %, based on 100 weight % of the first polyol.

Another objective of the present invention is to provide a shoe sole made from the polyurethane microcellular elastomer of the present invention.

Another objective of the present invention is to provide a use for the polyurethane microcellular elastomer of the present invention.

The present invention also provides for a method for preparing a polyurethane microcellular elastomer prepared from polycarbonate polyol obtained by reacting carbon dioxide and alkene oxide. This method can not only reduce the demand for raw materials derived from petrochemicals, but also realize the utilization of the “greenhouse gas” carbon dioxide. Furthermore, the present invention optimizes the amount of the polycarbonate polyol obtained by reacting carbon dioxide and alkene oxide used and adjusts the NCO content of isocyanate-terminated prepolymer so that the polyurethane microcellular elastomer prepared therefrom has good physical and mechanical properties.

### SPECIFIC MODE FOR CARRYING OUT THE INVENTION

In contrast to the conventional polyester polyols and/or polyether polyols produced through petrochemical routes, the present invention selects and further optimizes the polycarbonate polyol obtained by reacting carbon dioxide and alkene oxide to prepare polyurethane microcellular elastomer. In this invention, the polycarbonate polyol obtained by reacting carbon dioxide and alkene oxide can be (1) added in the first polyol for preparing the isocyanate-terminated prepolymer, (2) added in the second polyol for reacting with the isocyanate-terminated prepolymer, (3) added in the first polyol and the second polyol at the same time, by optimizing the amount of the polycarbonate polyol and adjusting the NCO content of isocyanate-terminated prepolymer, to obtain a polyurethane microcellular elastomer with good physical and mechanical properties for industrial applications.

CN1400229A discloses a method for preparing polyurethane rigid foam or polyurethane flexible foam through a one-step process by using polycarbonate polyol obtained by reacting carbon dioxide and alkene oxide as raw materials instead of conventional polyester polyols or polyether polyols. However, this method cannot be used to prepare polyurethane microcellular elastomer that meets industrially applicable standards. In the present invention, the prepolymer approach not only optimizes the reaction route, but also adjusts the amount of polycarbonate polyol obtained by a reaction of carbon dioxide and alkene oxide to prepare polyurethane microcellular elastomer with good mechanical and physical properties to meet the requirements for industrial application.

### Polyurethane Microcellular Elastomer

In one embodiment of the present invention, the polyurethane microcellular elastomer comprises the reaction product of:
a) an isocyanate-terminated prepolymer, wherein said isocyanate-terminated prepolymer is the reaction product of an isocyanate and a first polyol;
b) a second polyol; and
c) a chain extender having an average molecular weight of less than or equal to 800;
   wherein, said first polyol and/or said second polyol comprise(s) at least one polycarbonate polyol obtained by reacting a carbon dioxide with an alkene oxide, wherein said polycarbonate polyol has the general formula (I)

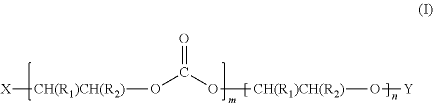

wherein,

R1 and R2
are independently selected from the group consisting of hydrogen, methyl, ethyl, and phenyl;

m is a natural number;

n is 0 or a natural number; and

X and Y
are independently a terminated group comprising hydroxyl.

In another embodiment of the present invention, the above polyurethane microcellular elastomer further comprises a blowing agent.

In another embodiment of the present invention, the above polyurethane microcellular elastomer further comprises a surfactant.

The above polyurethane microcellular elastomer can be prepared by reacting a), b) and c) in the presence of a catalyst.

In the present invention, polycarbonate polyol obtained by the reaction of carbon dioxide with an alkene oxide may be prepared according to the chemical process disclosed in US 2008/021154, WO 2006/103213, CN 1060299A, or CN 101024685A. The reaction of carbon dioxide with an alkene oxide can be carried out in the presence of starters and catalysts. The alkene oxide can be selected from, but is not limited to, ethylene oxide, 1,2-propylene oxide, 1,2-butylene oxide, 2,3-butylene oxide, 1,2-cyclohexene oxide, styrene oxide, or their mixtures. The starter can be selected from, but is not limited to, one or more polyols, and this one or more polyols can be selected from, but is not limited to, water, glycol, 1,2-propanediol, 1,3-propanediol, diethylene glycol, trimethylolpropane, or their mixtures. The catalyst can be selected from, but is not limited to, double metal cyanides (DMC), porphyrin metal catalysts, rare earth catalysts, or their mixtures. The reaction temperature can be selected from, but is not limited to, a temperature in the range of from 40 to 120° C. The pressure of carbon dioxide in the reaction can be selected from, but is not limited to, a pressure in the range of from 20 to 60 atm. The reaction time can be selected from, but is not limited to, an amount of time in the range of from 1 to 20 hours.

The isocyanate-terminated prepolymer a) is the reaction product of a polyisocyanate and a first polyol. The polyisocyanates and first polyols that can be used to prepare the isocyanate-terminated preoplymer a) are described in detail below in the section titled “Isocyanate-terminated prepolymer.”

The second polyol b) has an average molecular weight in the range of from 1,000 to 10,000, and an average functionality of from 1 to 5, preferably from 1.5 to 3.

The second polyol can comprise at least one polycarbonate polyol obtained by reacting carbon dioxide with an alkene oxide in an amount of from 5 to 100 weight %, preferably from 5 to 50 weight %, more preferably from 5 to 30 weight %, based on 100 weight % of the second polyol.

The second polyol can further comprise polyester polyols, polyether polyols, polycarbonate polyols, or their mixtures.

The polyester polyols can be prepared by reacting an organic dicarboxylic acid or dicarboxylic acid anhydride with a polyol. The dicarboxylic acid preferably comprises an aliphatic carboxylic acid containing from 2 to 12 carbon atoms, which can be selected from, but is not limited to, succinic acid, malonic acid, glutaric acid, adipic acid, suberic acid, azelaic acid, sebacic acid, decane-dicarboxylic acid, maleic acid, fumaric acid, phthalic acid, isophthalic acid, terephthalic acid, or their mixtures. The dicarboxylic acid anhydride can be selected from, but is not limited to, phthalic anhydride, terachlorophthalic anhydride, maleic anhydride, or their mixtures. The polyol can be selected from, but is not limited to, glycol, diethylene glycol, 1,2-propanediols, 1,3-propanediols, dipropylene glycol, 1,3-methylpropanediol, 1,4-butanediol, 1,5-pentanediol, 1,6-hexanediol, neopentyl glycol, 1,10-decanediol, glycerol, trimethylol-propane, or their mixtures. The polyester polyol can further comprise a polyester polyol prepared from a lactone. The lactone can be selected from, but is not limited to, ε-caprolactone.

The polyether polyols can be prepared by known processes, e.g., by reacting an alkene oxide with a starter in the presence of a catalyst. The catalyst can be selected from, but is not limited to, alkali hydroxides, alkali alkoxides, antimony pentachloride, boron fluoride etherate, or their mixtures. The alkene oxide can be selected from, but is not limited to, tetrahydrofuran, ethylene oxide, 1,2-propylene oxide, 1,2-butylene oxide, 2,3-butylene oxide, styrene oxide, or their mixtures. The starter can be selected from, but is not limited to, one or more polyols, and this one or more polyols can be selected from, but is not limited to, water, glycol, 1,2-propanediol, 1,3-propanediol, 1,4-butanediol, diethylene glycol, trimethylol-propane, or their mixtures.

The polycarbonate polyols can be prepared by reacting a diol with a dialkyl or diaryl carbonate or phosgene. The diol can be selected from, but is not limited to, 1,2-propanediol, 1,3-propanediol, 1,4-butanediol, 1,5-pentanediol, 1,6-hexanediol, diethylene glycol, trioxyethylene glycol, or their mixtures. The dialkyl or diaryl carbonate can be selected from, but is not limited to, diphenyl carbonate.

The chain extender c) is selected from compounds comprising active hydrogen atoms and having a molecular weight of less than 800, preferably in the range of from 18 to 400. The compounds comprising active hydrogen atoms can be selected from, but are not limited to, alkanediols, dialkylene glycols, polyalkylene polyols, or their mixtures, for example, glycol, 1,4-butanediol, 1,6-hexanediol, 1,7-heptanediol, 1,8-octanediol, 1,9-nonanediol, 1,10-decanediol, diethylene glycol, dipropylene glycol, polyoxyalkylene glycols, or their mixtures. The compounds comprising active hydrogen atoms can also comprise other grafted or unsaturated alkyl diols, or their mixtures, such as 1,2-propanediol, 2-methyl-1,3-propanediol, 2,2-dimethyl-1,3-propanediol, 2-butyl-2-ethyl-1,3-propanediol, 2-butylene-1,4-glycol, 2-butyne-1,4-glycol, alkanolamine, N-alkyl dialkanolamine. The N-alkyl dialkanolamine can be selected from, but is not limited to, ethanolamine, 2-propanolamine, 3-amido-2,2-dimethyl propyl alcohol, N-methyl diethanol amine, N-ethyl diethanol amine, or their mixtures. The compounds comprising active hydrogen atoms can also comprise an aliphatic amine, an aromatic amine, or their mixture. The aliphatic amine and/or aromatic amine can be selected from, but are not limited to, 1,2-ethylenediamine, 1,3-propylenediamine, 1,4-butylenediamine, 1,6-hexamethylenediamine, isophoronediamine, 1,4-cyclohexamethylenediamine, N,N′-diethyl-phenylenediamine, and 2,4-diaminotoluene, 2,6-diaminotoluene, or their mixtures.

The catalyst which can be used when preparing the polyurethane microcellular elastomer can be selected from, but is not limited to, amine catalysts, organometallic catalysts, or their mixtures. The amine catalysts can be selected from, but are not limited to, tertiary amines, triethylamine, tributylamine, N-ethylmorpholine, N,N,N′,N′-tetramethyl-ethylenediamine, pentamethyldiethylene-triamine, N,N-methylbenzylamine, N,N-dimethylbenzylamine, or their mixtures. The organometallic catalysts can be selected from, but are not limited to, organo-tin compounds, such as tin (II) acetate, tin (II) octoate, tin ethylhexonate, tin laurate, dibutyltin oxide, dibutyltin dichloride, dibutyltin diacetate, dibutyltin maleate, dioctyltin diacetate, or their mixtures.

The blowing agent can be selected from physical blowing agents or chemical blowing agents, preferably, but not limited to, water, halohydrocarbons, hydrocarbons, and gases. The halohydrocarbons can be selected from, but are not limited to, monochlorodifuloromethane, dichloromonofluoromethane, dichlorofluoromethane, trichlorofluromethane, or their mixtures. The hydrocarbons can be selected from, but are not limited to, butane, pentane, cyclopentane, hexane, cyclohexane, heptane, or their mixtures. The gases can be selected from, but are not limited to, air, CO₂, and N₂. Preferably, the blowing agent is water.

The surfactant can be selected from, but is not limited to, derivatives of siloxane.

### Isocyanate-Terminated Prepolymer

In another embodiment of the present invention, the isocyanate-terminated prepolymer a), which is used for preparing the polyurethane microcellular elastomer, comprises the reaction product of an isocyanate with a first polyol.

The first polyol comprises at least one polycarbonate polyol obtained by reacting carbon dioxide with an alkene oxide and the general formula of this polycarbonate polyol is

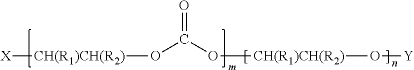

wherein,

R1 and R2
are independently selected from the group consisting of hydrogen, methyl, ethyl, and phenyl;

m is a natural number;

n is 0 or a natural number; and

X and Y
are independently a terminated group comprising hydroxyl.

The first polyol has an average molecular weight in the range of from 1,000 to 10,000 and a functionality of from 1 to 5, preferably from 1.5 to 3.

The first polyol can comprise at least one polycarbonate polyol obtained by reacting carbon dioxide with an alkene oxide in an amount of from 5 to 100 weight %, preferably from 5 to 50 weight %, more preferably from 5 to 30 weight %, based on 100 weight % of the second polyol.

The NCO content of the isocyanate-terminated prepolymer is from 5 to 25 weight %, based on 100 weight % of the isocyanate-terminated prepolymer.

The first polyol can further comprise polyester polyols, polyether polyols, polycarbonate polyols, or their mixtures.

The isocyanate has a general formula R(NCO)ₙ, wherein R is an aliphatic alkyl containing 2-18 carbon atoms, an aryl containing 6-15 carbon atoms, or an araliphatic alkyl containing 8-15 carbon atoms, and n is 2, 3, or 4.

The isocyanate can be selected from, but is not limited to, ethylene diisocyanate, 1,4-tetramethylene diisocyanate, hexamethylene diisocyanate (HDI), 1,2-dodecane diisocyanate, cyclobutane-1,3-diisocyanate, cyclohexane 1,3-diisocyanate, 1,4-diisocyanate, 1-isocyanato-3,3,5-trimethyl-5-isocyanatomethyl-cyclohexane, 2,4-hexahydrotoluene diisocyanate, hexahydro-1,3-phenylene diisocyanate, hexahydro-1,4-phenylene diisocyanate, perhydro-2,4-diphenylmethane diisocyanate, perhydro-4,4′-diphenylmethane diisocyanate, 1,3- phenylene diisocyanate, 1,4-phenylene diisocyanate, 1,4-durol diisocyanate, 1,4-stilbene diisocyanate, 3,3′-dimethyl-4,4′-biphenylene diisocyanate, toluene 2,4-diisocyanate (TDI), 2,6-diisocyanate (TDI), diphenylmethane-2,4′-diisocyanate (MDI), diphenylmethane-2,2′-diisocyanate (MDI), diphenylmethane-4,4′-diisocyanate (MDI), naphthylene-1,5-diisocyanate (NDI), their isomers, and mixtures thereof.

The isocyanate can also comprise a polyisocyanate modified with carbodiimide, uretoneimine, allophanate, or isocyanurate structures, preferably, but not limited to, diphenylmethane diisocyanate, diphenylmethane diisocyanate modified by carbodiimide, their isomers, and mixtures thereof.

### Preparation of Shoe Sole

In another embodiment of the present invention, the polyurethane microcellular elastomer can be used to manufacture footwear, such as shoe soles.

The examples and methods disclosed in this invention are illustrative and not limitative.

### Raw Materials Mentioned in the Context

Bayflex 2003E: polyester polyol, average molecular weight 2000, OH 56 mgKOH/g, available from BayerMaterialScience.

Arcol 1026: polyether polyol, average molecular weight 4000, OH 28 mgKOH/g, available from BayerMaterialScience.

Arcol 1362: polyether polyol, average molecular weight 6000, OH 28 mgKOH/g, available from BayerMaterialScience.

Hyperlite E851: polyether polyol, OH 18.5 mgKOH/g, BayerMaterialScience.

Dabco EG, Dabco S-25: tertiary amine catalysts, available from Air Products.

DC 193: siloxane surfactant, available from Air Products.

### EXAMPLES

In the following examples, the polycarbonate polyol was obtained by reacting carbon dioxide with propylene oxide. This polycarbonate polyol had a number average molecular weight of 2000 g/mol, a functionality of 2.0, and an OH value of 48 mgKOH/g.

The isocyanate index X is defined as

$$X ( \% ) = \frac{[ moles of NCO groups in Component A ] \times 100 \%}{[ moles of NCO reactive groups in Component B ]}$$

### Preparation of Isocyanate-Terminated Prepolymers

### Examples 1-4

4,4′-MDI and polyol in the amounts listed in Table 1 were charged into a reactor and reacted at 70° C. for two hours, then cooled to 65° C. Thereafter, carbodiimidized-MDI (CD-MDI) in the amounts listed in Table 1 was charged into the reactor and stirred for 30 min, then cooled to room temperature to obtain prepolymers A1-A4.

### Example 5

4,4′-MDI and polyol in the amount listed in Table 1 were charged into a reactor and reacted at 60° C. for two hours, then cooled to 50° C. Thereafter, carbodiimidized-MDI (CD-MDI) in the amount listed in Table 1 was charged into the reactor and stirred for 30 min, then cooled to room temperature to obtain the prepolymer A5.

TABLE 1
Preparation of isocyanate-terminated prepolymers

| Example | 1 | 2 | 3 | 4 | 5 |
|---|---|---|---|---|---|
| 4,4′-MDI (wt. %) | 56 | 60 | 60 | 60 | 66 |
| CD-MDI (wt. %) | 6 | 6 | 6 | 6 | 5 |
| Bayflex 2003E (wt. %) | 38.0 | 32.3 | 27.2 | 23.8 | — |
| Polycarbonate polyol (wt. %) | — | 1.7 | 6.8 | 10.2 | — |
| Arcol Polyol 1021 (wt. %) | — | — | — | — | 22 |
| Tripropylene glycol (wt. %) | — | — | — | — | 7 |
| NCO (wt. %) | 19.3 | 19.5 | 19.9 | 19.2 | 19.8 |
| Obtained prepolymers | A1 | A2 | A3 | A4 | A5 |

As shown in Table 1, polycarbonate polyol obtained by the reaction of carbon dioxide and an alkene oxide was not used to prepare the isocyanate-terminated prepolymers of Examples 1 and 5. Polycarbonate polyol obtained by the reaction of carbon dioxide and an alkene oxide was used to prepare the isocyanate-terminated prepolymers of Examples 2-4.

### Preparation of Polyurethane Microcellular Elastomers

The isocyanate-terminated prepolymers of Examples 1-5 were used as Component A, respectively.

The second polyols, chain extenders, blowing agents, catalysts, and surfactants were used as Component B.

### Example 6

As shown in Table 2, Component B was mixed with the isocyanate-terminated prepolymer A2 at 45° C. using a mechanical stirrer.

The mixture was poured into an aluminum folding mould heated to 50° C. The mould was closed and the foam was demoulded after 8 minutes to obtain a polyurethane microcellular elastomer. The physical and mechanical properties of the polyurethane microcellular elastomer are listed in Table 2.

### Example 7

As shown in Table 2, Component B was mixed with the isocyanate-terminated prepolymer A3 at 45° C. using a mechanical stirrer.

The mixture was poured into an aluminum folding mould heated to 50° C. The mould was closed and the foam was demoulded after 8 minutes to obtain a polyurethane microcellular elastomer. The physical and mechanical properties of the polyurethane microcellular elastomer are listed in Table 2.

### Example 8

As shown in Table 2, Component B was mixed with the isocyanate-terminated prepolymer A4 at 45 ° C. using a mechanical stirrer.

The mixture was poured into an aluminum folding mould heated to 50° C. The mould was closed and the foam was demoulded after 8 minutes to obtain a polyurethane microcellular elastomer. The physical and mechanical properties of the polyurethane microcellular elastomer are listed in Table 2.

TABLE 2
Preparation of polyurethane microcellular elastomers

| Examples | | 6 | 7 | 8 |
|---|---|---|---|---|
| Component A | | A2 | A3 | A4 |
| Component B | Bayflex 2003E (wt. %) | 91.0 | 91.0 | 91.0 |
| | Ethylene glycol (wt. %) | 6.7 | 6.7 | 6.7 |
| | water (wt. %) | 0.4 | 0.4 | 0.4 |
| | Dabco EG (wt. %) | 1.5 | 1.5 | 1.5 |
| | DC193 (wt. %) | 0.4 | 0.4 | 0.4 |
| Index X (%) | | 94 | 98 | 98 |
| Physical | Density (kg/m³) | 550 | 550 | 550 |
| properties | Tensile strength (MPa) | 4.3 | 4.6 | 2.5 |
| | Tear strength (90°) (kN/m) | 21.2 | 20.8 | 10.8 |
| | Tear strength (trouser) (kN/m) | 7.1 | 5.9 | 2.8 |
| | Elongation at break (%) | 431 | 331 | 292 |
| | Ross Flex (mm, room | 0.15 | 0.7 | — |
| | temperature, 40k cycles) | | | |
| | Hardness (Asker C) | 66 | 67 | 45 |
| | Abrasion (mm³) (7.5N half way) | 251 | 338 | — |

As shown in Table 2, the polyurethane microcellular elastomers of Examples of 6-8 possess good physical and mechanical properties.

### Examples 9-12

As shown in Table 3, Component B was mixed with the isocyanate-terminated prepolymer A1 at 45° C. using a mechanical stirrer.

The mixture was poured into an aluminum folding mould heated to 50° C. The mould was closed and the foam was demoulded after 8 minutes to obtain a polyurethane microcellular elastomer. The physical and mechanical properties of the polyurethane microcellular elastomer are listed in Table 3.

TABLE 3
Preparation of polyurethane microcellular elastomers

| Examples | | 9 | 10 | 11 | 12 |
|---|---|---|---|---|---|
| Component A | | A1 | A1 | A1 | A1 |
| Component B | Bayflex 2003E (wt. %) | 86 | 81 | 71 | 61 |
| | Polycarbonate polyol (wt. %) | 5 | 10 | 20 | 30 |
| | Ethylene glycol (wt. %) | 6.7 | 6.7 | 6.7 | 6.7 |
| | water (wt. %) | 0.4 | 0.4 | 0.4 | 0.4 |
| | Dabco EG (wt. %) | 1.5 | 1.5 | 1.5 | 1.5 |
| | DC193 (wt. %) | 0.4 | 0.4 | 0.4 | 0.4 |
| Index X (%) | | 94 | 94 | 94 | 100 |
| Physical | Density (kg/m3) | 550 | 550 | 550 | 550 |
| properties | Tensile strength (MPa) | 4.3 | 3.1 | 3.6 | 2.8 |
| | Tear strength (90°) (kN/m) | 22.9 | 20.2 | 19.2 | 17.3 |
| | Tear strength(trouser) (kN/m) | 7.6 | 6.9 | 6.3 | 4.6 |
| | Elongation at break (%) | 477 | 479 | 471 | 379 |
| | Ross Flex (mm, room | 0.1 | 0.1 | 0.1 | 2.7 |
| | temperature, 40 cycles) | | | | |
| | Hardness (ASKER C) | 67 | 63 | 63 | 61 |
| | Abrasion (mm3) (7.5N half | 332 | 366 | 382 | 576 |
| | way) | | | | |

As shown in Table 3, the polyurethane microcellular elastomers in Examples 9-12 were prepared from second polyols comprising polycarbonate polyols obtained by the reaction of carbon dioxide and an alkene oxide.

The polyurethane microcellular elastomers of Examples 9-12 possess good physical and mechanical properties.

### Example 13-15

As shown in Table 4, Component B was mixed with the isocyanate-terminated prepolymer A5 at 45° C. using a mechanical stirrer.

The mixture was poured into an aluminum folding mould heated to 50° C. The mould was closed and the foam was demoulded after 8 minutes to obtain a polyurethane microcellular elastomer. The physical and mechanical properties of the polyurethane microcellular elastomer are listed in Table 4.

TABLE 4
Preparation of polyurethane microcellular elastomers

| Examples | | 13 | 14 | 15 |
|---|---|---|---|---|
| Component A | | A5 | A5 | A5 |
| Component B | Arcol 1362 (wt. %) | 70 | 60 | 50 |
| | Hyperlite E851 (wt. %) | 10 | 10 | 10 |
| | Polycarbonate polyol (wt. %) | 10 | 20 | 30 |
| | 1,4-butane diol (wt. %) | 6.7 | 6.7 | 6.7 |
| | Ethylene glycol (wt. %) | 1.0 | 1.0 | 1.0 |
| | water (wt. %) | 0.8 | 0.8 | 0.8 |
| | Dabco S-25 (wt. %) | 1.5 | 1.5 | 1.5 |
| Index X (%) | | 90 | 94 | 96 |
| Physical | Density (kg/m³) | 350 | 350 | 350 |
| properties | Tensile strength (MPa) | 1.2 | 1.0 | 0.9 |
| | Tear strength (90°) (kN/m) | 6.9 | 6.1 | 5.6 |
| | Tear strength (trouser) | 2.2 | 3.0 | 2.4 |
| | (kN/m) | | | |
| | Elongation at break (%) | 266 | 170 | 268 |
| | Hardness (Asker C) | 33 | 34 | 30 |
| | Compression set [50%] (%) | 9.9 | 12.8 | 15.6 |

As shown in Table 4, the polyurethane microcellular elastomers in Examples 13-15 were prepared from second polyols comprising polycarbonate polyols obtained by the reaction of carbon dioxide and an alkene oxide

The polyurethane microcellular elastomers of Examples 9-12 possess good physical and mechanical properties.

Although the present invention is illustrated through the above Examples, it is not limited by them in any way. Without departing from the spirit and scope of this invention, those skilled in the art can make any modifications and alternatives.

1. A polyurethane microcellular elastomer comprising the reaction product of:
a) an isocyanate-terminated prepolymer, wherein said isocyanate-terminated prepolymer is the reaction product of an isocyanate and a first polyol;
b) a second polyol; and
c) a chain extender having an average molecular weight of less than or equal to 800;
wherein said first polyol and/or said second polyol comprise(s) at least one polycarbonate polyol obtained by reacting carbon dioxide with an alkene oxide, wherein said polycarbonate polyol has the general formula (I): 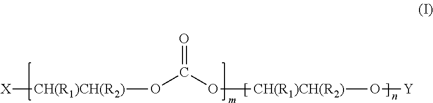
wherein,
R₁ and R₂
are independently selected from the group consisting of hydrogen, methyl, ethyl, and phenyl;
m is a natural number;
n is 0 or a natural number; and
X and Y
are independently a terminal group comprising
hydroxyl.

2. The polyurethane microcellular elastomer of claim 1, wherein said polyurethane microcellular elastomer comprises a surfactant.

3. The polyurethane microcellular elastomer of claim 1, wherein said alkene oxide is selected from the group consisting of ethylene oxide, 1,2-propylene oxide, 1,2-butylene oxide, 2,3-butylene oxide, 1,2-cyclohexene oxide, and styrene oxide.

4. The polyurethane microcellular elastomer of claim 1, wherein said first polyol and/or second polyol further comprise(s) a polyether polyol and/or a polyester polyol.

5. The polyurethane microcellular elastomer of claim 1, wherein the amount of said polycarbonate polyol obtained by reacting carbon dioxide with an alkene oxide in said first polyol is in the range of from 5 to 100 weight %, based on 100 weight % of said first polyol.

6. The polyurethane microcellular elastomer of claim 1, wherein the NCO content of said isocyanate-terminated prepolymer is in the range of from 5 to 25 weight %, based on 100 weight % of said isocyanate-terminated prepolymer.

7. The polyurethane microcellular elastomer of claim 1, wherein the amount of said polycarbonate polyol obtained by reacting carbon dioxide with alkene oxide in said second polyol is in the range of from 5 to 100 weight %, based on 100 weight % of said second polyol.

8. An isocyanate-terminated prepolymer, wherein said isocyanate-terminated prepolymer is the reaction product of an isocyanate with a first polyol, wherein said first polyol comprises at least one polycarbonate polyol obtained by reacting carbon dioxide with an alkene oxide, wherein said polycarbonate polyol has the general formula (I): 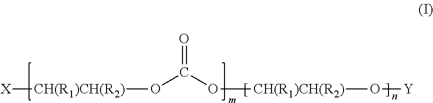
wherein,
R₁, R₂
are independently selected from the group consisting of hydrogen, methyl, ethyl, and phenyl;
m is a natural number;
n is 0 or a natural number; and
X and Y
are independently a terminal group comprising hydroxyl.

9. The isocyanate-terminated prepolymer of claim 8, wherein said alkene oxide is selected from the group consisting of ethylene oxide, 1,2-propylene oxide, 1,2-butylene oxide, 2,3-butylene oxide, 1,2-cyclohexene oxide, and styrene oxide.

10. The isocyanate-terminated prepolymer of claim 8, wherein the amount of said polycarbonate polyol obtained by reacting carbon dioxide with alkene oxide in said first polyol is in the range of from 5 to 100 weight %, based on 100 weight % of the first polyol.

11. The isocyanate-terminated prepolymer of claim 8, wherein the NCO content of said isocyanate-terminated prepolymer is in the range of from 5 to 25 weight %, based on 100 weight % of said isocyanate-terminated prepolymer.

12. The isocyanate-terminated prepolymer of claim 8, wherein said first polyol further comprises a polyether polyol and/or a polyester polyol.

13. A shoe sole comprising the polyurethane microcellular elastomer of claim 1.
